(19) United States
(12) Patent Application Publication
(10) Pub. No.: US 2019/0008733 A1
(43) Pub. Date: Jan. 10, 2019

(54) METHODS FOR REDUCING SWEAT AND/OR BODY ODOR, USING PHOSPHATE COMPOUNDS

(71) Applicant: Henkel AG & Co. KGaA, Duesseldorf (DE)

(72) Inventors: Bernhard Banowski, Duesseldorf (DE); Marcus Claas, Hilden (DE)

(73) Assignee: Henkel AG & Co. KGaA, Duesseldorf (DE)

(21) Appl. No.: 15/745,153

(22) PCT Filed: Apr. 26, 2016
(86) PCT No.: PCT/EP2016/059222

§ 371 Date: Jan. 16, 2018

(30) Foreign Application Priority Data
Jul. 16, 2015 (DE) 10 2015 213 344.3

(51) Int. Cl. A61K 8/24; A61K 8/26; A61K 8/28; A61Q 15/00

(52) U.S. Cl. CPC A61K 8/24; A61K 8/26; A61K 2800/88; A61Q 15/00; A61K 8/28

(57) ABSTRACT

The present disclosure relates to a method for reduction of perspiration of the body and/or reduction of the body odor released by the perspiration in which antiperspirant cosmetic agents containing phosphate (M1) are applied to the human skin and remain on the application point for at least about 1 hour. Use of at least one phosphate compound in this method has an antiperspirant effect or a reduction of the body odor released by sweat.

### CROSS-REFERENCE TO RELATED APPLICATION

The application is a U.S. National Stage entry under 35 U.S.C. § 371 based on International Application No. PCT/EP2016/059222, filed Apr. 26, 2016, which was published under PCT Article 21 (2) and which claims priority to German Application No 10 2015 213 344.3 filed, Jul. 16, 2015 which are all hereby incorporated in their entirety by reference.

### TECHNICAL FIELD

The present disclosure relates to a method for reduction of perspiration of the body and/or reduction of the body odor released by the perspiration in which an antiperspirant cosmetic agent containing phosphate (M1) is applied to the human skin and remains on the application point for at least 1 hour. This antiperspirant agent (M1) contains at most about 1 weight % antiperspirant aluminum and/or aluminum-zirconium salt. Use of at least one phosphate compound in the method has an antiperspirant effect or a reduction of the body odor released by sweat.

### BACKGROUND

The present disclosure also relates to the use of at least one phosphate compound for reduction of perspiration of the body and/or reduction of the body odor released by perspiration.

Washing, cleaning and care for the body are a basic human need and modern industry continuously attempts to meet these human needs in a variety of ways. Long-lasting elimination or at least reduction of the body odor and underarm wetness are especially important for daily hygiene. Numerous deodorizing or antiperspirant personal care products are known in the prior art, which were developed for use in body regions with a high density of sweat glands, particularly in the underarm region. They are assembled in a wide variety of dosage forms, such as a powder, stick form, aerosol spray, pump spray, liquid and gel-like roll-on application, lotion, gel and as a moist flexible substrate (deodorant wipes).

The cosmetic antiperspirants used in the method of the prior art for reduction of perspiration contain at least an antiperspirant compound, particularly in the form of halides and/or hydroxy halides of aluminum and/or zirconium. These antiperspirant compounds reduce the perspiration of the body by temporarily constricting and/or clogging the excretory ducts of the sweat glands such that the amount of sweat can be reduced by from about 20 to about 60 percent. Due to their antimicrobial effect, they also reduce the degradation of initially odorless sweat into malodorous compounds and thus the development of body odor.

However, the halides and/or hydroxy halides of aluminum and/or zirconium contained in the agents that are used can cause unpleasant skin reactions in combination with the acidic pH value of these agents. Use of the aforementioned antiperspirant compounds can also cause stains on the clothing.

Therefore, there is a need to replace the antiperspirant halides and/or hydroxy halides of aluminum and/or zirconium with other antiperspirant cosmetic active ingredients in the method for reduction of perspiration. These active ingredients should have a good antiperspirant effect, good compatibility with the skin and simple formulability. In addition, these antiperspirant active ingredients should not have a negative influence on the storage stability of the cosmetic agents that are used.

The task of the present disclosure is to provide a method for reduction of perspiration of the body and/or reduction of the body odor released by perspiration, which avoids and/or at least diminishes the disadvantages of the prior art and results in a reliable reduction of underarm wetness with simultaneously good compatibility with the skin.

### BRIEF SUMMARY

Methods of reducing sweat and/or body odor, and cosmetic agents for the same, are provided. In an exemplary embodiment, a method includes applying an antiperspirant cosmetic agent (M1) to the human skin, wherein the antiperspirant cosmetic agent (M1) remains on the application point for at least one hour. The antiperspirant cosmetic agent (M1) is in a cosmetically compatible carrier and includes, relative to the total weight of the cosmetic agent (M1), at least one phosphate compound and a maximum of about 1.0 wt. % of antiperspirant aluminum and/or aluminum zirconium salts.

In another embodiment, a package unit includes at least a first container and a second container. The first container includes a cosmetic agent (M1) that includes, in an acceptable carrier, at least one phosphate compound and at most about 1.0 wt. % antiperspirant aluminum and/or aluminum zirconium salt, relative to a total weight of the cosmetic agent (M1).

In yet another embodiment, an antiperspirant cosmetic agent (M1) includes a cosmetically acceptable carrier. The agent (M1) also includes at least one phosphate compound and at most about 1.0 wt. % antiperspirant aluminum and/or aluminum zirconium salt.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It was surprisingly discovered that the use of at least one phosphate compound in cosmetic methods resulted in an antiperspirant and/or odor-inhibiting effect that is almost comparable to the achieved antiperspirant and odor-inhibiting effect of aluminum and/or aluminum zirconium salts used in the method of the prior art. However, no skin irritation is observed with use of these phosphate compounds.

Therefore, the subject of the present disclosure is a cosmetic method for reduction of perspiration of the body and/or reduction of the body odor released by the perspiration in which an antiperspirant cosmetic agent (M1) is applied to the human skin and remains on the application point for at least one hour, wherein the antiperspirant cosmetic agent (M1) in a cosmetically compatible carrier—relative to the total weight of the cosmetic agent (M1)—

a) contains at least one phosphate compound and
b) a maximum of about 1.0 weight % of antiperspirant aluminum and/or aluminum zirconium salts.

Use of cosmetic agents containing phosphate as contemplated herein achieves an outstanding reduction of sweat, particularly underarm sweat, without the occurrence of skin irritations. A reduction in the development of body odor was also observed with the use of at least one phosphate compound. Use of even extremely small amounts and/or with the absence of antiperspirant halides and/or hydroxy halides of aluminum and/or zirconium also guarantees an effective reduction of underarm sweat and/or body odor. The cosmetic agents (M1) used as contemplated herein can be obtained by mixing at least one phosphate compound with at least one aluminum and/or aluminum zirconium salt.

As contemplated herein, the term “antiperspirant” should be understood as the reduction of perspiration of the sweat glands of the body. The term “aluminum and/or aluminum zirconium salts” in the scope of the present disclosure should be understood as comprising chloride, bromide and iodide of aluminum and/or zirconium, as well as compounds of the formulae Al(OH)_{y}X and Zr(OH)_{z}X, wherein X stands for a halide ion in the formula.

The term “phosphate compound” should be understood as including salts of ortho-phosphoric acid, such as orthophosphate (dihydrogen phosphates), secondary orthophosphates (hydrogen phosphates) and tertiary orthophosphate. The ortho-phosphoric acid does not contain any organic radicals. This term also comprises condensation products of salts of ortho-phosphoric acid which arise from hydrogen phosphates due to intermolecular dehydration with formation of P—O—P bridges. Therefore, the polyphosphates are composed exclusively of phosphorus and oxygen and do not contain any carbon-containing radicals. The polyphosphates of the present disclosure can be produced, for example, by a chemical reaction of ortho-phosphoric acid and sodium hydroxide and/or potassium hydroxide and subsequent dehydration and melting. The term “phosphate compound” should also be understood as comprising mixtures of salts of ortho-phosphoric acid and polyphosphates. However, the term “phosphate compound” according to the present disclosure should not be understood as comprising phosphoric acid esters, particularly esters of phosphoric acid with organic radicals.

According to the present disclosure, the term “cosmetic oil” relates to an oil that is suitable for cosmetic use, which is not miscible with water in all amounts. The cosmetic oil used as contemplated herein is neither fragrances nor essential oils.

The term “wax” in the scope of the present disclosure should be understood as substances which are kneadable or firm to stiff and brittle at about 20° C., have a rough to fine-crystalline structure and are transparent to opaque in color, but not glassy. These substances also melt at temperatures above about 25° C. without breaking down, are only slightly fluid above the melting point (less viscous), have a consistency and solubility that are highly temperature-dependent and are polishable under light pressure. The term “fatty acids” as used in the scope of the present disclosure should be understood as aliphatic carboxylic acids having unbranched or branched carbon radicals with from about 4 to about 40 carbon atoms. The fatty acids used in the scope of the present disclosure can be naturally occurring and synthetically manufactured fatty acids. Furthermore, the fatty acids can be mono- or polyunsaturated.

Finally, the term “fatty alcohols” in the scope of the present disclosure should be understood as aliphatic, monohydric, primary alcohols having unbranched or branched hydrocarbon radicals with from about 4 to about 40 carbon atoms. The fatty alcohols used in the scope of the present disclosure can also be mono- or polyunsaturated. The specification of wt. % presently relates to the total weight of the antiperspirant cosmetic agent used as contemplated herein (M1), unless something different is indicated.

As contemplated herein, the antiperspirant cosmetic agent (M1) is used advantageously for reduction of perspiration in the underarm and/or reduction of underarm odor. Therefore, preferred methods as contemplated herein are exemplified in that the antiperspirant cosmetic agent (M1) is applied to the skin of the armpit cavity. The antiperspirant cosmetic agent used as contemplated herein (M1) contains at least one phosphate compound in a cosmetic carrier. As contemplated herein, this carrier is water-free, aqueous, alcoholic or aqueous-alcoholic.

As contemplated herein, a water-free carrier should be understood to mean a carrier that contains less than about 10 wt. % free water relative to the total weight of the antiperspirant cosmetic agent (M1). Free water within the meaning of the present disclosure is understood to mean water that is different from crystal water, hydration water or similar molecularly bound water of the components that are used. The cosmetic carrier of the antiperspirant cosmetic agent used as contemplated herein (M1) preferably contains free water in a total amount of less than about 8.0 wt. %, preferably less than about 5.0 wt. %, more preferably less than about 3.0 wt. %, even more preferably less than about 1.0 wt. %, in particular about 0 wt. %, based on the total weight of the antiperspirant cosmetic agent (M1).

Water-free carriers preferably contain a cosmetic oil that is fluid at about 20° C. and about 1.013 hPa, which is selected from the group of (i) volatile cyclic silicone oils, particularly cyclic and linear silicone oils; (ii) volatile non-silicone oils, particularly liquid paraffin oils and isoparaffin oils; (iii) non-volatile silicone oils; (iv) non-volatile non-silicone oils; and (v) mixtures thereof.

The term “liquid oil” refers to oils as contemplated herein which have a vapor pressure of from about 2.66 Pa to about 40,000 Pa (from about 0.02 to about 300 mm Hg), preferably from about 10 to about 12,000 Pa (from about 0.1 to about 90 mm Hg), more preferably from about 13 to about 3,000 Pa (from about 0.1 to about 23 mm Hg), particularly from about 15 to about 500 Pa (from about 0.1 to about 4 mm Hg) at about 20° C. and an environmental pressure of about 1.013 hPa.

Furthermore, the term “non-liquid oils” within the meaning of the present disclosure is understood to mean oils that have a vapor pressure of less than about 2.66 Pa (about 0.02 mm Hg) at about 20° C. and an environmental pressure of about 1,013 hPa.

As contemplated herein, it can be preferable to use mixtures of volatile silicone oils and volatile non-silicone oils as a carrier, because a dryer skin feeling is achieved as a result. Furthermore, it may be preferable in the context of the present disclosure if a non-volatile silicone oil and/or a non-volatile non-silicone oil is used as carrier in order mask non-soluble components, such as talcum, phosphate compounds or ingredients dried on the skin.

As contemplated herein, use of mixtures of non-volatile and volatile cosmetic oils is particularly preferable, because this enables adaptation parameters such as skin feeling, visibility of residue and stability of the antiperspirant cosmetic agent used as contemplated herein (M1) and, therefore, the agent can be better adapted to the needs of the consumer.

The volatile and non-volatile silicone oils, as well as volatile and non-volatile non-silicone oils used in the context of the present disclosure are disclosed in the published patent applications DE 10 2010 063 250 A1 and DE 10 2012 222 692 A1.

The cosmetic oil that is liquid at about 20° C. and about 1,013 hPa is used in a total quantity of from about 1.0 to about 98 wt. %, preferably from about 2.0 to about 85 wt. %, more preferably from about 4.0 to about 75 wt. %, even more preferably from about 6.0 to about 70 wt. %, most preferably from about 8.0 to about 60 wt. %, particularly from about 8.0 to about 20 wt. %, relative to the total weight of the antiperspirant cosmetic agent (M1).

An aqueous carrier as contemplated herein contains at least about 10 wt. % free water relative to the total weight of the antiperspirant cosmetic agent. The carrier preferably contains free water in a total quantity of from about 15 to about 95 wt. %, preferably from about 30 to about 70 wt. %, particularly from about 40 to about 60 wt. %, relative to the total weight of the antiperspirant cosmetic agent (M1).

As contemplated herein, alcoholic carriers contain at least about 1.0 wt. % relative to the total weight of the antiperspirant cosmetic agent (M1), a C₁-C₄ alcohol and/or a C₂-C₆ alkyl alcohol having at least one hydroxy group. This includes, for example, ethanol, ethylene glycol, isopropanol, 1.2-propylene glycol, 1.3-propylene glycol, glycerin, n-butanol, 1.3-butylene glycol and mixtures thereof. Use of ethanol as an alcoholic carrier is especially preferred.

Therefore, a preferred alcoholic carrier contains ethanol in a total quantity of from about 1.0 to about 99 wt. %, preferably from about 5.0 to about 70 wt. %, preferably from about 7.0 to about 50 wt. %, particularly from about 10 to about 30 wt. %, relative to the total weight of the antiperspirant cosmetic agent (M1).

Aqueous-alcoholic carriers according to the present disclosure are understood to mean aqueous carriers containing at least about 1.0 wt. %, relative to the total weight of the antiperspirant cosmetic agent (M1), of a C₁-C₄ alcohol and/or a C₂-C₆ alkyl alcohol having at least one hydroxy group.

The antiperspirant and/or odor-inhibiting effect achieved by employing the method is preferably achieved exclusively by using at least one phosphate compound. Therefore, in the context of the present disclosure, it is advantageous if the cosmetic agent (M1) contains about 0 wt. % of antiperspirant aluminum and/or aluminum-zirconium salts relative to the total weight of the cosmetic agent (M1). As contemplated herein, therefore, the cosmetic agent (M1) that is used does not contain any antiperspirant salt of aluminum and/or aluminum-zirconium. It is preferable, in particular, that none of the antiperspirant salts of aluminum and/or aluminum-zirconium specified below are contained:

(i) aqueous astringent inorganic salts of aluminum, particularly aluminum chlorohydrate, aluminum sesquichlorohydrate, aluminum dichlorohydrate, aluminum hydroxide, potassium aluminum sulfate, aluminum bromohydrate, aluminum chloride, aluminum sulfate;
(ii) aqueous astringent organic salts of aluminum, particularly aluminum chlorohydrex propylene glycol, aluminum chlorohydrex polyethylene glycol, aluminum propylene glycol complex, aluminum sesquichlorohydrex propylene glycol, aluminum sesquichlorohydrex polyethylene glycol, aluminum propylene glycol dichlorohydrex, aluminum polyethylene glycol dichlorohydrex, aluminum undecylenoyl collagen amino acid, sodium aluminum lactate, sodium aluminum hydroxy lactate, aluminum lipo amino acids, aluminum lactate, aluminum chlorohydroxyallantoinate, sodium aluminum chlorohydroxy lactate;
(iii) aqueous astringent inorganic aluminum zirconium salts; particularly aluminum zirconium trichlorohydrate, aluminum zirconium tetrachlorohydrate, aluminum zirconium pentachlorohydrate, aluminum zirconium octachlorohydrate;
(iv) aqueous astringent organic aluminum zirconium salts, particularly aluminum zirconium propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine; as well as
(v) mixtures thereof.

In the context of the present disclosure, it has been found to be advantageous to use linear and/or cyclic phosphate compounds. Linear phosphate compounds can be salts of orthophosphoric acid and linear or chain-shaped polyphosphates. The latter are also known as catena-polyphosphates. Ring-shaped polyphosphates that are also identified as meta- or cyclo-polyphosphates can be used as cyclic phosphate compounds. Therefore, preferred methods as contemplated herein are exemplified in that the at least one phosphate compound is selected from linear or cyclic phosphate compounds and mixtures thereof.

Use of linear phosphate compounds of formula (I) has been found to be particularly advantageous with regard to the antiperspirant and/or odor-inhibiting effect. Therefore, preferred methods as contemplated herein are exemplified in that the at least one phosphate compound has the formula (I)

MₓH_{y}P_{z}O_{3z+1} (I),

where
x and z, each independently of one another, denote integers from about 1 to about 50, preferably from about 1 to about 20, more preferably from about 1 to about 10, most preferably from about 1 to about 4,
y denotes integers from about 0 to about 100, preferably from about 0 to about 40, more preferably from about 0 to about 20, most preferably from about 0 to about 4, and
M denotes a monovalent or polyvalent cation from the group of alkali metals, alkaline earth metals, transition metals, organic cations or aluminum. As contemplated herein, the term “organic cations” is understood to mean cationic compounds which contain at least one carbon atom.

In this connection, it is preferable as contemplated herein if the organic cation is selected from the group of basic amino acids, compounds of the formula N⁺(R)₄, where R denotes linear or branched C₂-C₁₀ alkyl groups, which are substituted with at least one hydroxyl group, imidazolium compounds, pyridinium compounds, pyrrolidinium compounds and sulfonium compounds. Basic amino acids have at least one proton acceptor in the side chain. Examples of such amino acids are arginine, histidine and lysine. Compounds of the formula N⁺(R)₄ are understood to mean protonated alkylamines and alkanolamines. Particularly suitable alkylamines are monoalkyl, dialkyl and trialkylamines, such as diethyl and triethylamine, diisopropylamine and isopropylamine. Alkanolamines or amino alcohols have both an amino group and a hydroxyl group.

Therefore, the radical R in the formula N⁺(R)₄ contains at least one OH group. In the context of the present disclosure, suitable alkanolamines include choline, which is also known by the name (2-hydroxyethyl)trimethyl ammonia and has the formula HO—CH₂—CH₂—N⁺(CH₃)₃. Additional suitable alkanolamines are selected from the group of 2-aminoethane-1-ol (monoethanolamine), 3-aminopropane-1-ol, 4-amino-butane-1-ol, 5-aminopentane-1-ol, 1-aminopropane-2-ol (monoisopropanolamine), 1-aminobutane-2-ol, 1-aminopentane-2-ol, 1-aminopentane-3-ol, 1-aminopentane-4-ol, 2-amino-2-methyl-propanol, 2-amino-2-methylbutanol, 3-amino-2-methylpropane-1-ol, 1-amino-2-methylpropane-2-ol, 3-aminopropane-1-diol, 2-amino-2-methylpropane-1,3-diol, 2-amino-2-ethyl-1,3-propandiol, N,N-dimethyl-ethanolamine, triethanolamine, diethanolamine and triisopropanolamine. The imidazolium compounds (formula (III)), pyridinium compounds (formula IV)), pyrrolidinium compounds (formula (V)) and sulfonium compounds (formula (VI)) used in the context of the present disclosure preferably have the formula (III) to (VI) indicated below, where the radical R₁ denotes hydrogen, OH or a linear or branched C₂-C₁₀ alkyl group, which is also substituted with at least one hydroxyl group

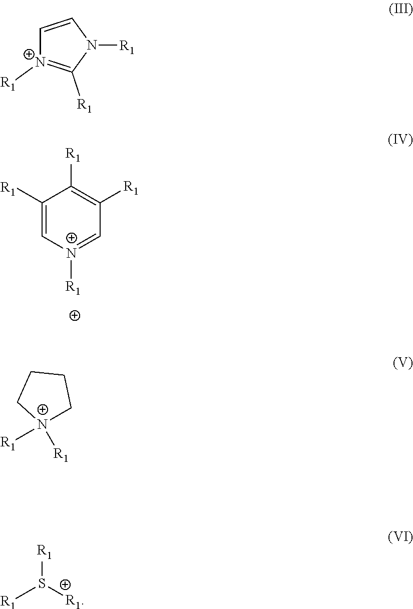

If mono-, di-, tri- and tetraphosphate are used as a phosphate compound of formula (I), the use of specific inorganic cations has shown to be advantageous with regard to the antiperspirant and/or odor-inhibiting effect. Therefore, preferred methods are exemplified in that x and z in formula (I), each independently of each other, denote the integers from about 1 to about 4, y denotes integers from about 0 to about 4 and M denotes Na⁺, K+, Li+, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴⁺ or ammonia.

Especially good results were achieved in the context of the method if sodium hydrogen phosphate of the formula NaH₂PO₄ or sodium pyrophosphate of the formula Na₂H₂P₂O₇ is used as a phosphate compound. Therefore, preferred methods are exemplified in that x denotes the integer about 1 or about 2, y denotes the integer about 2, z denotes the integer about 1 and M denotes Na⁺ in formula (I). The use of cosmetic agents that contain sodium hydrogen phosphate and/or sodium pyrophosphate in the method achieves an especially good antiperspirant and/or odor-inhibiting effect, even with the absence of the aforementioned aluminum and/or aluminum zirconium salts. Furthermore, the sodium hydrogen phosphate and/or sodium pyrophosphate has good skin compatibility.

However, in the context of the present disclosure, it is also possible to use a cyclic phosphate compound. Use of the cyclic phosphate compound can take place on its own or in combination with a linear phosphate compound. As contemplated herein, it is preferable if the at least one phosphate compound has formula (II)

M′(PO₃)ₙ (II)

where
n denotes integers from about 2 to about 10, preferably from about 2 to about 8, more preferably from about 2 to about 6, most preferably from about 2 to about 4, and
M′ denotes a monovalent or polyvalent cation from the group of alkali metals, alkaline earth metals, transition metals, organic cations or aluminum.

In this connection, it is preferable as contemplated herein if the organic cation is selected from the group of basic amino acids, compounds of the formula N⁺(R)₄, where R denotes linear or branched C₂-C₁₀ alkyl groups, which are substituted with at least one hydroxyl group, imidazolium compounds, pyridinium compounds, pyrrolidinium compounds and sulfonium compounds. Preferred organic cations have been described above in connection with the organic cations of formula (I).

Especially good results are achieved if a methophosphate which has an inorganic cation as a cation is used as a phosphate compound of formula (II). Therefore, preferred methods as contemplated herein are exemplified in that n denotes the integer 3 and M′ denotes Na⁺, K+, Li+, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴⁺ or ammonia in formula (II).

An especially preferred embodiment of the present disclosure uses antiperspirant cosmetic agents which contain at least one special phosphate compound. Therefore, it is especially preferred that the cosmetic agent (M1) contains at least one phosphate compound of the formula NaH₂PO₄ and/or the formula Na₂H₂P₂O₇ and/or the formula M′(PO₃)ₙ, where n denotes the integer 3 and M′ denotes Na⁺, K⁺, Li⁺, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴ or ammonia. Use of sodium hydrogen phosphate of the formula NaH₂PO₄ and/or sodium pyrophosphate of the formula Na₂H₂P₂O₇ and/or metaphosphates in the method as contemplated herein result in an especially good reduction of perspiration and/or body order released by perspiration.

In the context of the present disclosure it is advantageous to use the at least one phosphate compound in the cosmetic agent (M1) in specific quantity ranges. Therefore, preferred methods as contemplated herein are exemplified in that the cosmetic agent (M1) contains the at least one phosphate compound in a total quantity of from about 0.05 to about 90 wt. %, preferably from about 0.1 to about 40 wt. %, more preferably from about 1.0 to about 25 wt. %, and most preferably from about 2.0 to about 20 wt. %, relative to the total weight of the cosmetic agent (M1). Use of the at least one phosphate compound, particularly the formula NaH₂PO₄ and/or Na₂H₂P₂O₇ and/or the formula M′(PO₃)ₙ where n denotes the integer 3 and M′ denotes Na⁺, K⁺, Li⁺, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴⁺ or ammonia in the aforementioned total quantities, achieves an outstanding reduction of perspiration and/or body odor released by perspiration with the method as contemplated herein. Furthermore, no skin irritation occurs with use of the at least one phosphate compound in the aforementioned quantities.

The antiperspirant cosmetic agent (M1) used in the method preferably has a specific pH value. Stable formulation of the antiperspirant cosmetic agents (M1) used as contemplated herein is possible within this range without undesired interactions occurring between the ingredients. Furthermore, no skin irritation occurs with use of these agents with these pH values. Therefore, it is advantageous if the antiperspirant cosmetic agent (M1) has a pH value from about pH 2 to about pH 10. Adjustment of the desired pH value can take place with conventional acids and bases known to a person skilled in the art and in antiperspirant cosmetics.

The antiperspirant cosmetic agent (M1) used in the method can contain other substances in addition to the aforementioned ingredients.

As contemplated herein, the antiperspirant cosmetic agent (M1) preferably contains at least one wide additive selected from the group of (i) emulsifiers and/or surfactants; (ii) thickening agents; (iii) chelating agents; (iv) deodorant active ingredients; (v) polyethylene glycols; (vi) skin-cooling active ingredients; (vii) pH control agents; (viii) skin-care active ingredients, such as moisturizers, skin-calming substances, skin-lightening substances, skin-smoothing substances; (ix) waxes; (x) preservatives; and (xi) mixtures thereof.

As contemplated herein, suitable emulsifiers and surfactants are preferably selected from anionic, cationic, non-ionic, amphoteric, particularly ampholytic and zwitterionic emulsifiers and surfactants. Surfactants are amphiphilic (bifunctional) compounds that include at least one hydrophobe and at least one hydrophile molecular part. The hydrophobic radical is preferably a hydrocarbon chain with from about 8 to about 28 carbon atoms, which can be saturated or unsaturated, linear or branched. It is especially preferable if this C₈-C₂₈ aklyl chain is linear. Emulsifiers and surfactants usable in the context of the present disclosure are, for example, disclosed in published patent applications DE 10 2012 222 692 A1, DE 10 2010 063 250 A1 and DE 10 2010 055 816 A1.

Use of substances selected from cellulose ethers, xanthan gum, sclerotium gum, succinoglucanen, polygalactomannans, pectins, agar, carrageenan (carrageenan), tragacanth, gum arabic, gum karaya, tara gum, gellan gum, gelatin, propylene glycol alginate, alginic acids and their salts, polyvinyl pyrrolidones, polyvinyl alcohols, polyacrylic amides, physically (for example by pregelatinization) and/or chemically modified starches, acrylic acid-acrylate copolymers, acrylic acid-acrylamide copolymers, acrylic acid-vinyl pyrrolidone copolymers, acrylic acid-vinyl formamide-copolymers and polyacrylates is preferred for the thickening of the antiperspirant cosmetic agent (M1) used as contemplated herein. Cellulose ethers such as carboxymethyl celluloses are especially preferred for use as a thickening agent. Furthermore, especially preferred thickening agents are selected from carbomers. Carbomers are thickening cross-linked polymers made of acrylic acid, methacrylic acid and salts thereof. The cross-linking can take place by employing polyfunctional compounds, such as polyaklyl ether of polysaccharides or polyalcohols, such as sucrose allyl ether, pentaerythritol allyl ether or propylene allyl ether. Homopolymers of acrylic acid or salts thereof, which are cross-linked with a pentaerythritol allyl ether, a sucrose allyl ether or a propylene allyl ether, are preferred in the context of the present disclosure. A usable thickening agent in the context of the present disclosure is a copolymer including of C₁₀₋₃₀ acrylic acrylate, acrylic acid, methacrylic acid and esters thereof, which is cross-linked with a sucrose allyl ether or a pentaerythritol allyl ether. Thickening agents based on carbomers include the products available under the trade name Carbopol® (BF Goodrich, Ohio, USA), such as Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol ultrez 10, Carbopol ultrez 20, or Carbopol ultrez 21.

Furthermore, lipophile thickening agents can be used to thicken the antiperspirant cosmetic agents (M1) used as contemplated herein. Lipophile thickening agents preferred as contemplated herein are selected from hydrophobic clay materials, bentonites, hectorites, fumed silica and derivatives thereof.

The antiperspirant cosmetic agents (M1) used as contemplated herein may contain at least one chelating agent in a total quantity of from about 0.01 to about 3.0 wt. %, preferably from about 0.02 to about 1.0 wt. %, particularly from about 0.05 to about 0.1 wt. %, relative to the total weight of the antiperspirant cosmetic agent (M1) as an additive. In the context of the present disclosure, preferred chelating agents are selected from the group of beta-alaninediacetic acid, cyclodextrin, diethylene triamine pentamethylene phosphonic acid, sodium, potassium, calcium disodium, calcium-, ammonium and triethanolamine salts of ethylenediaminetetraacetic acid (EDTA), etidronic acid, hydroxyethylethylenediaminetetraacetic acid (HEDT) and sodium salts thereof, sodium salts of nitrilotriacetic acid (NTA), diethylene triamine pentaacetic acid, phytic acid, hydroxypropyl cyclodextrin, methylcyclodextrin, aminotrimethylene phosphonate pentasodium, ethylenediamine tetramethylene phosphonate pentasodium, diethylene triamine pentaacetate pentasodium, pentasodium triphosphate, potassium EDTMP, sodium EDTMP, sodium dihydroxyethylglycinate, sodium phytate, sodium polydimethylglycinylphenol sulfonate, tetrahydroxyethyl ethylenediamine, tetrahydroxypropyl ethylenediamine, tetrasodium iminodisuccinate, trisodium ethylenediamine disuccinate, tetrasodium-n,n-bis(carboxymethyl)glutamate, tetrasodium-DL-alanine-n,n-diacetate and desferrioxamine.

The deodorizing effect of the antiperspirant cosmetic agents (M1) used as contemplated herein can be enhanced, if at least one deodorant active ingredient having an antibacterial and/or bacteriostatic and/or enzyme-inhibiting and/or odor-neutralizing and/or odor-absorbing effect in a total amount of from about 0.0001 to about 40 wt. %, preferably from about 0.2 to about 20 wt. %, preferably from about 1 to about 15 wt. %, particularly from about 1.5 to about 5.0 wt. %, based on the total weight of the antiperspirant cosmetic agent (M1), is included. If ethanol is used in the agents used as contemplated herein, it does not apply as a deodorant active ingredient in the context of the present disclosure; rather, it is a component of the carrier. Examples of deodorant active ingredients preferred as contemplated herein are disclosed in published patent application DE 10 2010 063 250 A1.

Furthermore, preferred antiperspirant cosmetic agents (M1) used as contemplated herein can contain at least one water-soluble polyethylene glycol with from about 3 to about 50 ethylene oxide units. Water-soluble polyethylene glycols which can preferably be used are described, for example in the published patent application DE 10 2010 063 250 A1.

In addition, the antiperspirant cosmetic agents (M1) used as contemplated herein may also contain at least one skin-cooling active ingredient. Suitable skin-cooling active ingredients as contemplated herein are, for example, menthol, isopulegol and menthol derivatives, such as menthyl lactate, menthyl glycolate, menthyl ethyl oxamates, menthyl pyrrolidone carboxylic acid, menthyl methyl ether, menthoxypropane diol, menthoxyglycerol acetal (9-methyl-6-(1-methylethyl)-1,4-dioxaspiro (4.5) decane-2-methanol), monomenthyl succinate, 2-hydroxymethyl-3,5,5-trimethylcyclohexanol and 5-methyl-2-(1-methylethyl) cyclohexyl-n-ethyl oxamate. Menthol, isopulegol, menthyl lactate, menthoxypropane diol, menthyl pyrrolidone carboxylic acid and 5-methyl-2-(1-methylethyl)cyclohexyl-n-ethyl oxamate and mixtures of these substances, in particular mixtures of menthol and menthyl lactate, menthol, menthol glycolate and menthyl lactate, menthol and menthoxypropanediol or menthol and isopulegol, are preferred as skin-cooling active ingredients.

As contemplated herein, acids and/or alkalizing agents and/or buffers are preferred for use as pH control agents. As contemplated herein, inorganic acids are preferred for use as acids (such as hydrochloric acid, sulfuric acid or phosphoric acid) or organic acids (such as citric acid, tartaric acid or malic acid). Alkalizing agents usable as contemplated herein are preferably selected from the group comprising ammonia, basic amino acids, alkali hydroxides, alkaline earth metal hydroxides, carbonates and hydrogen carbonates, alkanolamines, for example amino-2-methyl-1-propanol, monoethanolamine, triethanolamine, diethanolamine and triisopropanolamine, alkali metal metasilicates, urea, morpholine, n-methylglucamine and imidazole. Lithium, sodium and potassium, particularly sodium or potassium are preferred for use as alkali metal ions. Carbonic acid bicarbonate buffer, carbonic acid silicate buffer, acetic acid acetate buffer, ammonia buffer, citric acid or citrate buffer, buffer based on tris(hydroxymethyl)-aminomethane, buffer based on 4-(2-hydroxyethyl)-1-piperazineethanesulfonic acid, buffer based on 4-(2-hydroxyethyl)-piperazine-1-propanesulfonic acid, buffer based on 2-(n-morpholino)ethanesulfonic acid and barbital acetate buffer, in particular, are suitable as buffer systems in the context of the present disclosure. Selection of the appropriate buffer system is determined on the basis of the desired pH value of the antiperspirant cosmetic agent (M1) used as contemplated herein. However, the at least one phosphate compound used as contemplated herein does not fall under the pH control agents listed above in the form of acids and buffer systems.

Furthermore, the antiperspirant cosmetic agents (M1) used as contemplated herein can contain at least one wax. This wax is preferably selected from the group of (i) fatty acid glycerol mono-, di- and triesters; (ii) butyrospermum parkii (shea butter); (iii) esters of monohydric C₈₋₁₈ alcohols with saturated C₁₂₋₁₈ monocarboxylic acids; (iv) linear, primary C_{12-C24}-alkanols; (v) esters of a saturated, monohydric C₁₆₋₆₀ alkanol and a saturated C_{8-C36} monocarboxylic acid; (vi) glycerol triesters of saturated linear C₁₂₋₃₀ carboxylic acids, which can be hydroxylated, such as glycerol esters of hydrogenated vegetable oils; (vii) natural vegetable waxes; (viii) animal waxes; (ix) synthetic waxes; and (x) mixtures thereof. Waxes usable in the context of the present disclosure are disclosed in published patent application DE 10 2012 222 692 A1.

The wax is preferably used in a total quantity of from about 0.01 to about 60 wt. %, preferably from about 3.0 to about 40 wt. %, more preferably from about 5.0 to about about 30 wt. %, particularly from 6.0 to about 25 wt. %, relative to the total weight of the antiperspirant cosmetic agent (M1).

Furthermore, as contemplated herein, it is preferable if the antiperspirant cosmetic agent (M1) used as contemplated herein also contains at least one preservative. Preservatives preferred as contemplated herein are formaldehyde separators iodopropynylbutyl carbamates, parabens, phenoxyethanol, ethanol, benzoic acid and the salts thereof, dibromodicyanobutane, 2-bromo-2-nitro-propane-1,3-diol, imidazolidinylurea, 5-chloro-2-methyl-4-isothiazolin-3-one, 2-chloroacetamide, benzalkonium chloride, benzyl alcohol, salicylic acid and salicylates. Additional usable preservatives in the context of the present disclosure are the substances listed in Annex 6 of the Cosmetics Regulation, as well as cosmetic raw materials with preserving characteristics or raw materials that support or enhance the preserving effect of the aforementioned preservatives. The preservatives are preferably included in a total quantity of from about 0.01 to about 10 wt. %, preferably from about 0.1 to about 7.0 wt. %, more preferably from about 0.2 to about 5.0 wt. %, particularly from about 0.3 to about 2.0 wt. %, relative to the total weight of the antiperspirant cosmetic agent (M1).

In the context of the present disclosure, it is preferable if the antiperspirant cosmetic agent (M1) used as contemplated herein is present as a water-in-oil emulsion. In particular, this can be a sprayable water-in-oil emulsion, which can be sprayed by employing a propellant. If the antiperspirant cosmetic agents (M1) used as contemplated herein contain a propellant, it is preferably included in a total quantity of from about 1 to about 98 wt. %, preferably from about 20 to about 90 wt. %, more preferably from about 30 to about 85 wt. %, particularly from about 40 to about 75 wt. %, relative to the total weight of the antiperspirant cosmetic agent (M1). Preferred propellants (propellant gases) are propane, propene, n-butane, iso-butane, iso-butene, n-pentane, pentene, iso-pentane, iso-pentene, methane, ethane, dimethylester, nitrogen, air, oxygen, nitrous oxide, 1,1,1,3-tetrafluorethane, heptafluoro-n-propane, perfluoroethane, monochlorodifluormethane, 1,1-difluorethane and tetrafluoropropenes, both individually and in mixtures thereof. Hydrophilic propellant gases, such as carbon dioxide, can also be used advantageously in the context of the present disclosure if the proportion of hydrophilic gases is selected to be low and lipophilic propellant gas (such as propane/butane) is present in excess. Propane, n-butane, iso-butane and mixtures of these propellant gases are especially preferred. It has been found that the use of n-butane as the only propellant gas can be particularly preferred as contemplated herein.

As contemplated herein, however, it can be preferred in equal measure if the antiperspirant cosmetic agent used as contemplated herein (M1) is present as an oil-in-water emulsion. In this case, the cosmetic agent is preferably sprayed as a propellant-free pump spray or squeeze spray or applied as a roll-on.

The application of the antiperspirant cosmetic agent (M1) used as contemplated herein can take place by employing different methods. According to a preferred embodiment, the antiperspirant cosmetic agent (M1) used as contemplated herein is packaged as a spray application. The spray application is carried out with a spray device containing a filling in a container of the liquid, viscous flowable, suspension-like or powdery antiperspirant cosmetic agent (M1). The filling can be carried out under the pressure of a propellant, as described above, (compressed gas cans, compressed gas packs, aerosol packs), or it can be a pump atomizer without propellant gas (pump spray/squeeze bottle) to be operated mechanically. The atomization of the antiperspirant cosmetic agent used in the method as contemplated herein (M1) can be carried out physically, mechanically or electromechanically, for example by employing Piezo effects or electric pumps. Containers and withdrawal devices which can be used in the context of this embodiment are described, for example, in the published patent application DE 10 2012 222 692 A1. The at least one phosphate compound preferably has an average particle size D₅₀ of from about 1 to about 300 μm in these sprayable cosmetic agents (M1), preferably from about 5 to about 125 m, particularly from about 10 to about 100 m.

Furthermore, the antiperspirant cosmetic agent (M1) used as contemplated herein can be preferentially packaged as a stick, soft solid, lotion, gel, roll-on or loose or compact powder. Formulation of the antiperspirant cosmetic agent (M1) used as contemplated herein in a specific dosage form, such as an antiperspirant roll-on, an antiperspirant stick or an antiperspirant gel is preferentially based on the requirements of the intended use. Therefore, depending on the intended use, the antiperspirant cosmetic agents (M1) used as contemplated herein can be produced in a solid, semi-solid manner, liquid, dispersed, emulsified, suspended, gel-like, multi-phase or powder form. For the purposes of the present disclosure, the term “liquid” also encompasses any types of solid-state dispersions in liquids. Furthermore, in the context of the present disclosure, multi-phase antiperspirant cosmetic agents (M1) used as contemplated herein are understood to mean agents which have at least two different phases with a phase separation and in which the phases are arranged horizontally, in other words one above the other, or vertically, that is to say next to one another. Application can, for example, take place with a roller ball applicator, a pump atomizer or with a solid stick. If the antiperspirant cosmetic agents (M1) used as contemplated herein are in the form of solid sticks, it is preferred if the at least one phosphate compound has an average particle size D₅₀ from about 1 to about 300 m, preferably from about 5 to about 125 m, more preferably from about 10 to about 100 m, particularly from about 3 to about 20 m.

It can likewise be preferred within the scope of the present disclosure if the antiperspirant cosmetic agent (M1) is included on and/or in a disposable substrate, selected from the group of wipes, pads and dabbers. Particular preference is given to wet wipes, in other words wet wipes prefabricated for the user, preferably packaged individually, which, for example, are well known from the field of glass cleaning or from the field of moist toilet papers. Such wet wipes, which can advantageously also contain preservatives, are coated with an antiperspirant cosmetic agent (M1) used as contemplated herein and are preferably individually packaged. Preferable substrate materials are selected from porous flat cloths. These cloths include cloths made of woven and non-woven (fleece) synthetic and natural fibers, felt, paper or foam, such as hydrophilic polyurethane foam. Preferred deodorizing or antiperspirant substrates as contemplated herein can be produced by saturation or impregnation or also by melting an antiperspirant cosmetic agent (M1) used as contemplated herein on to a substrate.

Especially preferred embodiments AF 1 to AF 29 of the cosmetic agents (M1) in the method as contemplated herein are listed in the following tables (all specifications are made in wt. %, insofar as nothing different is specified):

| | AF 1 | AF 2 | AF 3 | AF 4 |
|---|---|---|---|---|
| Phosphate compound | 0.01-40 | 0.2-30 | 1.0-25 | 2.0-20 |
| Aluminum and/or | 0-1.0 | 0-0.5 | 0-0.2 | 0 |
| aluminum zirconium salt | | | | |
| Cosmetic carrier ¹⁾ | Ad 100 | Ad 100 | Ad 100 | Ad 100 |
| | AF 2 | AF 3 | AF 4 | AF 5 |
| Phosphate compound of | 0.01-40 | 0.2-30 | 1.0-25 | 2.0-20 |
| formula (I) ²⁾ | | | | |
| Aluminum and/or | 0-1.0 | 0-0.5 | 0-0.2 | 0 |
| aluminum zirconium salt | | | | |
| Cosmetic carrier ¹⁾ | Ad 100 | Ad 100 | Ad 100 | Ad 100 |
| | AF 6 | AF 7 | AF 8 | AF 9 |
| Phosphate compound of | 0.01-40 | 0.2-30 | 1.0-25 | 2.0-20 |
| formula (II) ³⁾ | | | | |
| Aluminum and/or | 0-1.0 | 0-0.5 | 0-0.2 | 0 |
| aluminum zirconium salt | | | | |
| Cosmetic carrier ¹⁾ | Ad 100 | Ad 100 | Ad 100 | Ad 100 |
| | AF 10 | AF 11 | AF 12 | AF 13 |
| Phosphate compound of | 0.01-40 | 0.2-30 | 1.0-25 | 2.0-20 |
| formula (I) ²⁾ and | | | | |
| formula (II) ³⁾ | | | | |
| Aluminum and/or | 0-1.0 | 0-0.5 | 0-0.2 | 0 |
| aluminum zirconium salt | | | | |
| Cosmetic carrier ¹⁾ | Ad 100 | Ad 100 | Ad 100 | Ad 100 |
| | AF 14 | AF 15 | AF 16 | AF 17 |
| Na₂H₂P₂O₇ and/or | 0.01-40 | 0.2-30 | 1.0-25 | 2.0-20 |
| NaH₂PO₄ | | | | |
| Aluminum and/or | 0-1.0 | 0-0.5 | 0-0.2 | 0 |
| aluminum zirconium salt | | | | |
| Cosmetic carrier ¹⁾ | Ad 100 | Ad 100 | Ad 100 | Ad 100 |
| | AF 18 | AF 19 | AF 20 | AF 21 |
| Phosphate compound | 0.01-40 | 0.2-30 | 1.0-25 | 2.0-20 |
| Aluminum and/or | 0-1.0 | 0-0.5 | 0-0.2 | 0 |
| aluminum zirconium salt | | | | |
| Emulsifier | 0.1-15 | 0.5-10 | 1.0-8.0 | 2.0-6.0 |
| Cosmetic carrier ¹⁾ | Ad 100 | Ad 100 | Ad 100 | Ad 100 |
| | AF 22 | AF 23 | AF 24 | AF 25 |
| Phosphate compound of | 0.01-40 | 0.2-30 | 1.0-25 | 2.0-20 |
| formula (I) ²⁾ and | | | | |
| formula (II) ³⁾ | | | | |
| Aluminum and/or | 0-1.0 | 0-0.5 | 0-0.2 | 0 |
| aluminum zirconium salt | | | | |
| Emulsifier | 0.1-15 | 0.5-10 | 1.0-8.0 | 2.0-6.0 |
| Cosmetic carrier ¹⁾ | Ad 100 | Ad 100 | Ad 100 | Ad 100 |
| | AF 26 | AF 27 | AF 28 | AF 29 |
| Na₂H₂P₂O₇ and/or | 0.01-40 | 0.2-30 | 1.0-25 | 2.0-20 |
| NaH₂PO₄ | | | | |
| Aluminum and/or | 0-1.0 | 0-0.5 | 0-0.2 | 0 |
| aluminum zirconium salt | | | | |
| Emulsifier | 0.1-15 | 0.5-10 | 1.0-8.0 | 2.0-6.0 |
| Cosmetic carrier ¹⁾ | Ad 100 | Ad 100 | Ad 100 | Ad 100 |

¹⁾ water-free, aqueous or aqueous-alcoholic carrier,
²⁾ x and z, each independently of each other, denote the integers from about 1 to about 4, y denotes integers from about 0 to about 4 and M denotes Na⁺, K+, Li+, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴⁺ or ammonia,
³⁾ n denotes the integer 3 and M′ denotes Na⁺, K⁺, Li⁺, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴⁺ or ammonia,
⁴⁾ emulsifier selected from the group of ethoxylated C₁₂-C₁₈ alkanols with from about 10 to about 30 mole of ethylene oxide per mole of alkanol, ethoxylated C₈-C₂₄ carboxylic acids with from about 10 to about 30 mole of ethylene oxide per mole of carboxylic acid, C₈-C₂₂ alkyl mono- and oligoglycosides, ethoxylated sterols having a degree of ethoxylation of more than about 5, partial esters of polyglycerols having from about 2 to about 10 glycerol units and from about 1 to about 4 saturated or unsaturated, linear or branched, optionally hydroxylated C₈-C₃₀ carboxylic acid residues, in particular ethoxylated C₁₂-C₁₈ alkanols with from about 10 to about 30 mole of ethylene oxide per mole of alkanol

In the context of the present disclosure, it can also be stipulated that an additional cosmetic agent (M2) containing at least one antiperspirant active ingredient is applied. In the process, the agents (M1) and (M2) can be applied in succession in any arbitrary sequence or simultaneously. In this connection, it can be stipulated that the cosmetic agent (M1) is applied first and then cosmetic agent (M2). However, it is also possible to apply cosmetic agent (M2) first and then cosmetic agent (M1). Furthermore, the cosmetic agent (M1) and cosmetic agent (M2) are applied simultaneously. The time span between the use of the two agents (M1) and M2) is preferably from about 0 seconds (simultaneous application) to about 24 hours.

Therefore, in the context of the present disclosure, it is preferred if an additional cosmetic agent (M2) containing at least one antiperspirant aluminum and/or aluminum zirconium salt in a cosmetically acceptable carrier is applied to the skin. In the process, the cosmetic agent (M2) differs from the cosmetic agent (M1). Consequently, the antiperspirant effect of the method can be further improved.

If an additional cosmetic agent (M2) should be used in the method as contemplated herein, it is advantageous to store the individual cosmetic agents (M1) and (M2) in separate containers.

Therefore, a further subject of the present disclosure is a package unit (kit-of-parts), comprising—separately packaged—

a) at least one first container (C1) containing a cosmetic agent (M1) comprising, in a cosmetically acceptable carrier, at least one phosphate compound and at most about 1.0 wt. %, relative to the total weight of the cosmetic agent (M1), antiperspirant aluminum and/or aluminum-zirconium salts, and
b) at least one second container (C2) containing a cosmetic agent (M2) comprising at least one antiperspirant active ingredient.

As contemplated herein, the term “antiperspirant active ingredient” is understood to mean active ingredients which lessen or reduce the perspiration of the sweat glands of the body, wherein, however, the phosphate compounds contained in the agent (M1) do not fall under these active ingredients. In the context of the present disclosure, the antiperspirant aluminum and/or aluminum zirconium salts used as an antiperspirant active ingredient in the cosmetic agent (M2) in connection with the first subject of the present disclosure are preferred.

With regard to the cosmetic agent (M1) in the container (C1) applies mutatis mutandis to the cosmetic agents (M1) used in the method.

Finally, an additional subject of the present disclosure also relates to the use of at least one phosphate compound for reduction of perspiration of the body and/or reduction of the body odor released by perspiration.

With regard to the phosphate compound used as contemplated herein, the method and the packaging unit as contemplated herein apply mutatis mutandis.

The following examples explain the present disclosure without limiting it.

### EXAMPLES

### In-Vivo Test for Antiperspirant Effect

In order to determine the antiperspirant effect, an antiperspirant study was carried out on the back of 16 test subjects. For this purpose, the following antiperspirant agents were used:

| Antiperspirant agent | No. |
|---|---|
| Aqueous solution with 10% ACH*, pH 4 | V-I |
| Aqueous solution with 6.5 wt. % of phosphate compound^{a)}, pH 4 | E-I** |
| Aqueous solution with 12 wt. % of phosphate compound^{a)}, pH 4 | E-II** |

*active substance
**as contemplated herein
^{a)}sodium pyrophosphate of the formula Na₂H₂P₂O₇ and/or sodium hydrogen phosphate of the
formula NaH₂PO₄

In each case, about 40 μL of the antiperspirant agent V-I and about 75 μL of the cosmetic agents E-I and E-II were applied to the back of 16 test subjects on one side next to the backbone After about 5 minutes, the treated areas were covered with an occlusive non-adsorbing film. These non-absorbing pads were removed after about 2 hours. The compositions were applied to the back of the test strip on the back of the test tape on four successive days in each case in the manner described above. About 24 h after the last application of the composition, absorbent pads were applied to the areas on the back of the protuberances, where the compositions have been previously applied. Furthermore, the pads were also applied at the same height on the other side of the backbone as a control group. After the test subjects had sweated at about 80° C. in the sauna for about 15 minutes, the amount of the sweat collected by the pads was determined gravimetrically, wherein each composition has been compared to the respectively corresponding untreated area on the back.

The sweat reduction was determined from the gravimetric determination of the welding quantity, wherein all determined values were statistically significant.

The sweat reduction of each composition in comparison with an untreated area of the skin is shown in the following table:

| No. | Sweat reduction |
|---|---|
| V-I | 50% |
| E-I | 18% |
| E-II | 25% |

The use of cosmetic agents containing special phosphate compounds leads to a reduction in perspiration and reduced body odor when these agents are applied to the skin.

### 2. Formulations

The phosphate compound used in the following examples is preferably sodium pyrophosphate of the formula Na₂H₂P₂O₇ and/or sodium hydrogen phosphate of the formula NaH₂PO₄. Cosmetic agents used as contemplated herein (quantitative data in wt. %)

| | 1 | 2 | 3 | 4 | 5 | 6 |
|---|---|---|---|---|---|---|
| Stearyl Alcohol | 24.0 | 24.0 | 24.0 | 18 | 18 | 18 |
| Novata AB | — | — | — | 4 | 4 | 4 |
| Phosphate compound | 22.0 | 30.0 | 22.0 | 17.6 | 17.6 | 17.6 |
| PPG-14 Butyl Ether | 10.0 | 10.0 | 10.0 | 15.3 | 15.3 | 15.3 |
| Hardened castor oil (e.g. | 3.0 | 3.0 | 3.0 | 1.5 | 1.5 | 1.5 |
| cutina HR) | | | | | | |
| Myristyl myristate | 1.5 | 1.5 | 1.5 | — | — | — |
| DL menthol | 0.2 | 0.2 | 0.2 | — | — | — |
| Eucalyptol | 0.2 | 0.2 | 0.2 | — | — | — |
| Anethol | 0.2 | 0.2 | 0.2 | — | — | — |
| Silica dimethyl silylate | 1.4 | 1.4 | 1.4 | — | — | — |
| Silica | 0.3 | 0.3 | 0.3 | — | — | — |
| Talcum | — | — | — | 3 | 3 | 3 |
| Emulgin B1 | — | — | — | 3 | 3 | 3 |
| Perfume | 2.0 | 2.0 | 2.0 | 1 | 1 | 1 |
| Cyclomethicone (at least | ad | ad | ad | ad | ad | ad |
| 95 wt. % cyclopentadienyl) | 100 | 100 | 100 | 100 | 100 | 100 |

Antiperspirant cosmetic agents used as contemplated herein in the form of an oil-in-water emulsion (quantitative data in wt.-%)

| | 7 | 8 |
|---|---|---|
| Cutina ® AGS | 2.5 | 2.5 |
| Cutina ® FS45 | 3.5 | 3.5 |
| Eumulgin ® B2 | 0.8 | 0.8 |
| Eumulgin ® B3 | 0.8 | 0.8 |
| Diisopropyl adipate | 6.0 | 6.0 |
| Novata ® AB | 4.0 | 4.0 |
| Cutina ® CP | 5.0 | 5.0 |
| Cutina ® HR | 4.0 | 4.0 |
| Kester wax K62 | 5.0 | 5.0 |
| Phosphate compound | 10 | 40 |
| Talcum Pharma G | 10 | 10 |
| Perfume | 1.2 | 1.2 |
| 2-Benzylheptane-1-ol | — | 0.3 |
| Sensiva SC 50 | 0.6 | 0.6 |
| 1,2-Propanediol | 10 | 10 |
| Water, fully-demineralized | ad 100 | ad 100 |

Antiperspirant cosmetic agents used as contemplated herein in the form of a microemulsion (indications in % by wt. %)

| | 9 | 10 | 11 | 12 |
|---|---|---|---|---|
| Plantar ® 1200 | 1.7 | 1.7 | — | — |
| Plantar ® 2000 | 1.1 | 1.4 | 2.4 | 2.4 |
| Glycerol monooleate | 0.71 | 0.71 | — | — |
| Dioctyl ether | 4.0 | 4.0 | 0.090 | 0.090 |
| Octyldodecanol | 1.0 | 1.0 | 0.020 | 0.020 |
| Perfume oil | 1.0 | 1.0 | 1.0 | 1.0 |
| Phosphate compound | 20.0 | 10.0 | 5.0 | 5.0 |
| 1,2-Propylene glycol | 5.0 | 5.0 | — | — |
| Glycerol | — | — | 5.0 | 5.0 |
| 2-Benzylheptane-1-ol | 0.50 | — | — | — |
| Triethyl citrate | — | 0.50 | 0.50 | 0.50 |
| Water | ad 100 | ad 100 | ad 100 | ad 100 |

Antiperspirant cosmetic agents used as contemplated herein in the form of roll-ons (indications in % by wt. %)

| | 13 | 14 | 15 | 16 |
|---|---|---|---|---|
| Ethanol 96%, (DEP denatured) | 30 | 30 | 28 | 28 |
| Mergital ® CS 11 | 2.0 | 2.0 | — | — |
| Eumulgin ® B3 | 2.0 | 2.0 | 2.0 | 2.0 |
| Emulgin ® B1 | — | — | 2.0 | 2.0 |
| Phosphate compound | 40 | 15 | 14 | 10 |
| Hydroxyethylcellulose | 0.50 | 0.50 | 0.30 | 0.30 |
| EDTA | — | — | — | 0.050 |
| Cocamidopropyl PG-dimonium | 0.20 | — | — | — |
| chloride phosphate | | | | |
| Perfume oil | 0.80 | 0.80 | 1.0 | 1.0 |
| Water | ad 100 | ad 100 | ad 100 | ad 100 |

Antiperspirant cosmetic agents used as contemplated herein in the form of a water-in-oil emulsion (quantitative data in wt. %)

| | 21 | 22 |
|---|---|---|
| Phosphate compound | 15 | 30 |
| 1,2-Propylene glycol | 13.0 | 13.0 |
| Cyclohexsiloxane | 6.00 | 6.00 |
| Finsolv TN | 8.00 | 8.00 |
| Abil EM 90 | 1.20 | 1.20 |
| Polyethylene wax | 10.0 | 10.0 |
| (MW = 500 g/mole, Smp = 83 bis 91° C.) | | |
| Polyalphaolefin wax | 0.100 | 0.100 |
| (MW = 1800 g/mole, Smp = 41° C.) | | |
| EDTA | — | 0.0500 |
| Perfume | 1.00 | 1.00 |
| Water | Ad 100 | Ad 100 |

Cosmetic agents used as contemplated herein (quantitative data in wt. %)

| | 23 | 24 |
|---|---|---|
| Cyclopentasiloxane | 14.0 | 14.0 |
| Abil EM 97 | 3.00 | 3.00 |
| Ethanol 96% | 10.0 | 10.0 |
| Phosphate compound | 30.0 | 10.0 |
| 1,2-Propylene glycol | 20.3 | 20.3 |
| EDTA | — | 0.0750 |
| Perfume | 1.00 | 1.00 |
| Water | ad 100 | ad 100 |

Antiperspirant cosmetic agents used as contemplated herein (quantitative data in wt. %, relative to the total weight of the propellant-free composition)

| | 27 | 28 | 29 | 30 |
|---|---|---|---|---|
| Phosphate compound | 28.6 | 14.29 | 32.11 | 28.57 |
| Bentone38 VCG | 5.00 | 3.93 | 4.00 | 5.00 |
| Propylene carbonate | 1.50 | 0.71 | 1.50 | 1.80 |
| Fragrance | 7.14 | 6.50 | 5.00 | 6.50 |
| 2-Ethylhexyl palmitate | — | 73.57 | — | — |
| Isopropyl myristate | 7.37 | — | 10.00 | 12.0 |
| Triethyl citrate | — | — | 10.5 | 12.0 |
| Dimethicone 2-10 cst | — | — | — | 10.0 |
| C₁₀-C₁₃ Isoalkane | — | — | 35.39 | — |
| Cyclomethicone D5 | Ad 100 | Ad 100 | Ad 100 | Ad 100 |

The example compositions 27 to 30 were filled into an aluminum spray can optionally coated with epoxy-phenolic paint in a weight ratio of blowing agent (butane/propane/isobutane mixture) to a suspension of about 80:20 or about 85:15 or about 60:40 or about 90:10.

Antiperspirant cosmetic agents used as contemplated herein (quantitative data in wt. %, relative to the total weight of the propellant-free composition)

| | 31 | 32 | 33 |
|---|---|---|---|
| Phosphate compound | 15.0 | 10.0 | 30.0 |
| C₁₀-C₁₃ Isoalkane | 8.90 | 8.90 | 8.90 |
| Dow Corning ES-5227 DM | 1.40 | 1.40 | 1.40 |
| Isoceteth-20 | 0.500 | 0.500 | — |
| Dimethicone | 4.20 | 4.20 | 4.20 |
| Isopropyl myristate | 9.00 | 9.00 | 9.00 |
| 1,2 Propanediol | 7.00 | 25.0 | 25.0 |
| Phenoxyethanol | 0.500 | 0.500 | 0.500 |
| Perfume | 2.50 | 2.50 | 2.50 |
| L-Menthol | 0.400 | 0.300 | — |
| trans-Anethol | — | 0.300 | — |
| Eucalyptol | — | 0.300 | — |
| Perfume | 0.5 | 1.0 | 5.0 |
| Water | ad 100 | ad 100 | ad 100 |

The example compositions 31 to 33 were filled into an aluminum spray can optionally coated with epoxy-phenolic paint in a weight ratio of blowing agent (butane/propane/isobutane mixture) to a W/O emulsion of about 80:20 or about 85:15 or about 60:40 or about 90:10.

Antiperspirant cosmetic agents used as contemplated herein in the form of O/W emulsions (quantitative data indications in wt.)

| | 34 | 35 | 36 |
|---|---|---|---|
| Phosphate compound | 40.0 | 13.0 | 13.0 |
| Talc | 1.0 | — | — |
| Bentonite | 3.0 | — | — |
| Hectorite | — | — | 5.00 |
| Brij S 2 | 2.50 | 2.50 | 2.50 |
| Brij S 721 | 1.50 | 1.50 | 1.50 |
| Perfume | 1.10 | 1.10 | 1.10 |
| Arlamol E | 0.500 | 0.500 | 0.500 |
| Bisabolol | 0.100 | — | — |
| Dry Flo PC | 0.100 | — | 0.100 |
| Dow Corning 2501 Cosmetic Wax | 0.100 | — | 0.100 |
| Tocopheryl acetate | 0.100 | 0.100 | 0.100 |
| Water | ad 100 | ad 100 | ad 100 |

Antiperspirant cosmetic agents used as contemplated herein (quantitative data in wt. %, relative to the total weight of the propellant-free composition)

| | 37 | 38 | 39 | 40 |
|---|---|---|---|---|
| Phosphate compound | 33.0 | 33.0 | 33.0 | 33.0 |
| Cyclomethicone | 12.0 | 9.40 | — | — |
| C₁₀-C₁₃ Isoalkane | — | — | 9.40 | 8.90 |
| Dow Corning ES-5227 DM | — | 1.40 | 1.40 | 1.40 |
| Abil EM 90 | 3.00 | — | — | — |
| Brij IC 20 | — | — | — | 0.500 |
| Dimethicone | — | 4.20 | 4.20 | 4.20 |
| Isopropyl myristate | 9.00 | 9.00 | 9.00 | 9.00 |
| 1,2 Propanediol | 7.00 | 7.00 | 7.00 | 7.00 |
| Phenoxyethanol | 0.500 | 0.500 | 0.500 | 0.500 |
| Perfume | 2.50 | 2.50 | 2.50 | 2.50 |
| Water | add 100 | add 100 | add 100 | add 100 |

The example compositions 37 to 40 were filled into an aluminum spray can optionally coated with epoxy-phenolic paint in a weight ratio of blowing agent (butane/propane/isobutane mixture) to a W/O emulsion of about 80:20 or about 85:15 or about 60:40 or about 90:10.

Antiperspirant cosmetic agents used as contemplated herein in the form of W/O emulsions (quantitative data indications in wt. %)

| | 41 | 42 | 43 | 44 |
|---|---|---|---|---|
| Phosphate compound | 15 | 20 | 10 | 15 |
| Propylene glycol | 5.00 | 5.00 | 7.50 | 9.50 |
| C₁₂-C₁₅ Alkyl benzoate | 8.04 | 8.04 | 8.04 | 8.04 |
| Dimethicone 2 cst | 6.43 | 6.43 | 6.43 | 6.43 |
| Dimethicone 5 cst | 1.57 | 1.57 | 1.57 | 1.57 |
| Polyethylene | 10.2 | 11.7 | 9.70 | 12.2 |
| Abil EM 90 | 0.998 | 0.998 | 0.998 | 0.998 |
| Abil EM 97 | 1.22 | 1.22 | 1.22 | 1.22 |
| Synthetic wax | 0.100 | 0.100 | 0.100 | 0.100 |
| Perfume | 1.50 | 1.50 | 1.20 | 1.50 |
| Demineralized water | Ad 100 | Ad 100 | Ad 100 | Ad 100 |

Antiperspirant cosmetic agents used as contemplated herein in the form of water-free suspensions (indications in % by wt. %)

| | 45 | 46 | 47 | 48 |
|---|---|---|---|---|
| Bentone 38 V (hydrophobic) | 2.0 | 3.0 | — | — |
| Propylene carbonate | 1.50 | 2.0 | — | — |
| Ethylhexylpalmitate | 20.0 | 10.0 | — | — |
| Finsolv TN | — | 10.0 | 20.0 | 10.0 |
| Aerosil 972 V (hydrophobic) | — | — | 3.0 | 1.0 |
| Aerosil 200 | — | — | — | 1.0 |
| Protectate MOD 3 | — | 0.5 | — | 0.3 |
| Phosphate compound | 40.0 | 15.0 | 30.0 | 10.0 |
| Perfume | 1.0 | 1.0 | 2.0 | 2.0 |
| Cyclopentasiloxane | Ad 100 | Ad 100 | Ad 100 | Ad 100 |

Antiperspirant cosmetic agents used as contemplated herein in the form of PIT pump atomizer emulsions (quantitative data indications in wt. %)

| | 49 | 50 | 51 | 52 |
|---|---|---|---|---|
| Eumulgin BA 10 | 3.8 | 3.5 | — | — |
| Cetiol PGL | 5.0 | 4.5 | — | — |
| Cetiol OE | 10 | 10 | — | — |
| Glycerin 86% | 5.0 | 5.0 | — | — |
| Eumulgade SE | — | — | 4.5 | 5.0 |
| Eumulgin B2 | — | — | 1.5 | 2.0 |
| Cetiol S | — | — | 15 | 15 |
| Myritol 318 | — | — | 5.0 | 5.0 |
| Propylene glycol 1,2 | — | — | 5.0 | 5.0 |
| Panthenol | — | — | 0.2 | 0.5 |
| Perfume | 1.0 | 1.0 | 1.0 | 1.0 |
| Phosphate compound | 10 | 20 | 15 | 30 |
| Water | Ad 100 | Ad 100 | Ad 100 | Ad 100 |

Antiperspirant cosmetic agents used as contemplated herein in the form of a microemulsion (quantitative data in % by wt. %)

| | 53 | 54 | 55 | 56 |
|---|---|---|---|---|
| Plantaren 1200 | 1.7 | 1.8 | 2.0 | 1.7 |
| Plantaren 2000 | 1.2 | 1.4 | 2.4 | 1.2 |
| Glycerol monooleate | 0.7 | 0.7 | — | 0.7 |
| Dioctyl ether | 4.0 | 4.0 | 0.1 | 4.0 |
| Octyldodecanol | 1.0 | 1.0 | 0.1 | 1.0 |
| Perfume | 1.0 | 1.0 | 1.5 | 1.0 |
| Phosphate compound | 5.0 | 10.0 | 15.0 | 20.0 |
| Propylene glycol 1,2 | 5.0 | 5.0 | — | 5.0 |
| Water | Ad 100 | Ad 100 | Ad 100 | Ad 100 |

The following commercial products were used:

| | | Supplier/ |
|---|---|---|
| Commercial product | INCI | manufacturer |
| Abil EM 90 | CETYL PEG/PPG-10/1 Dimethicone | Evonik |
| Abil EM 97 | Bis-PEG/PPG-14/14 Dimethicone, | Evonik |
| | Cyclomethicone | |
| Arlamol E | PPG-15 Stearyl ether | Croda |
| Aerosil 200 | SILICA | Evonik |
| Aerosil 972 | SILICA DIMETHYL SILYLATE | Evonik |
| Bentone38 V CG | Disteardimonium Hectorite | Elementis |
| | | Specialities |
| Brij IC 20 | Isoceteth-20 | Croda |
| Brij S 2 | Steareth-2 | Croda |
| Brij S 721 | Steareth-21 | Croda |
| Cetiol PGL | HEXYLDECANOL, HEXYLDECYL | BASF |
| | LAURATE | |
| Cetiol OE | DICAPRYLYL ETHER | BASF |
| Cetiol S | DIETHYLHEXYLCYCLOHEXANE | BASF |
| Cutina ® AGS | GLYCOL DISTEARATE | BASF |
| Cutina ® CP | Cetyl Palmitate | BASF |
| Cutina ® FS45 | Palmitic Acid, Stearic Acid | BASF |
| Cutina ® HR | Hydrogenated Castor Oil | BASF |
| Dow Corning ® 2501 | Bis-PEG-18 Methyl ether dimethyl silane | Dow Corning |
| Dow Corning ES-5227 | Dimethicone, PEG/PPG-18/18 Dimethicone | Dow Corning |
| DM | in a weight ratio of 3:1 | |
| Dry Flo PC | Aluminum Starch Octenylsuccinate | National Starch |
| Emulgade SE | GLYCERYL STEARATE, CETEARETH- | BASF |
| | 20, CETEARETH-12, CETEARYL | |
| | ALCOHOL, CETYL PALMITATE | |
| Eumulgin ® B1 | Ceteareth-12 | BASF |
| Eumulgin ® B2 | Ceteareth-20 | BASF |
| Eumulgin ® B3 | Ceteareth-30 | BASF |
| Eumulgin ® BA 10 | BEHENETH-10 | BASF |
| Kester wax K62 | Cetearyl Behenate | Koster Keunen |
| Finsolv TN | C12-15 Alkyl Benzoate | Innospec |
| Mergital ® CS 11 | Ceteareth-11 | BASF |
| Myritol 318 | CAPRYLIC/CAPRIC TRIGLYCERIDE | BASF |
| Novata ® AB | Cocoglycerides (melting point 30-32° C.) | BASF |
| Plantar ® 1200 | LAURYL GLUCOSIDE, approx. 50% AS | BASF |
| Plantar ® 2000 | DECYL GLUCOSIDE, approx. 50% AS | BASF |
| Protectate MOD 3 | 2-BENZYLHEPTANOL, | Symrise |
| | PHENOXYETHANOL, HELIOTROPINE | |
| Sensiva ® SC 50 | 2-Ethylhexyl glycerin ether | Schülke & Mayr |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic method for reduction of perspiration of the body and/or reduction of the body odor released by the perspiration comprising:
applying an antiperspirant cosmetic agent (M1) to the human skin wherein the antiperspirant cosmetic agent (M1) remains on the application point for at least one hour, wherein the antiperspirant cosmetic agent (M1) in a cosmetically compatible carrier—relative to the total weight of the cosmetic agent (M1)—comprises:
a) at least one phosphate compound and
b) a maximum of about 1.0 weight % of antiperspirant aluminum and/or aluminum zirconium salts.

2. The method according to claim 1, wherein the cosmetic agent (M1) comprises about 0 wt. % of antiperspirant aluminum and/or aluminum-zirconium salts, relative to the total weight of the cosmetic agent (M1).

3. The method according to claim 1, wherein the at least one phosphate compound is selected from linear or cyclic phosphate compounds and mixtures thereof.

4. The methods according to claim 1 wherein the at least one phosphate compound has the formula (I)
MₓH_{y}P_{z}O_{3z+1} (I),
where
x and z, each independently of one another, denote integers from 1 to about 50;
y denotes integers from 0 to about 100; and
M denotes a monovalent or polyvalent cation selected from the group of alkali metals, alkaline earth metals, transition metals, organic cations or aluminum.

5. The method according to claim 4, wherein M denotes the organic cation, and wherein the organic cation is selected from the group of basic amino acids, choline, compounds of the formula NR₃, where R denotes linear or branched C₂-C₁₀ alkyl groups, which are substituted with at least one hydroxyl group, imidazolium compounds, pyridinium compounds, pyrrolidinium compounds and sulfonium compounds.

6. The method according to claim 4, wherein x and z in formula (I), each independently of each other, denote the integers from 1 to 4, y denotes integers from 0 to 4 and M is selected from the group of Na⁺, K+, Li+, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴⁺ or ammonia.

7. The method according to claim 4, wherein x denotes the integer 1 or 2, y denotes the integer 2, z denotes the integer 1 and M denotes Na⁺ in formula (I).

8. The method according to claim 1, wherein the at least one phosphate compound has formula (II)
M′(PO₃)ₙ (II), where
n denotes integers from 2 to 10; and
M′ denotes a monovalent or polyvalent cation selected from the group of alkali metals, alkaline earth metals, transition metals, organic cations or aluminum.

9. The method according to claim 8, wherein M′ denotes the organic cation, and wherein the organic cation is selected from the group of basic amino acids, compounds of the formula N⁺(R)₄, where R denotes linear or branched C₂-C₁₀ alkyl groups, which are substituted with at least one hydroxyl group, imidazolium compounds, pyridinium compounds, pyrrolidinium compounds and sulfonium compounds.

10. The method according to claim 8, wherein n denotes the integer 3 and M′ is selected from the group of Na⁺, K+, Li+, ½ Mg²⁺, ½ Ca²⁺, ½ Zn²⁺, ½ Mn²⁺, ½ Cu²⁺, ⅓ Fe³⁺, ¼ Zr⁴⁺, 1/4 Ti⁴⁺ or ammonia in formula (II).

11. The method according to claim 1, wherein the cosmetic agent (M1) comprises at least one phosphate compound of the formula NaH₂PO₄ and/or the formula Na₂H₂P₂O₇ and/or the formula M′(PO₃)ₙ, where n denotes the integer 3 and M′ is selected from the group of Na⁺, K⁺, Li⁺, ½ Mg²⁺, ½ Ca²⁺, ½ Zn²⁺, ½ Mn²⁺, ½ Cu²⁺, ⅓ Fe³⁺, ¼ Zr⁴⁺, ¼ Ti⁴⁺ or ammonia.

12. The methods according to claim 1, wherein the cosmetic agent (M1) comprises the at least one phosphate compound in a total quantity of from about 0.05 to about 90 wt. %, relative to the total weight of the cosmetic agent (M1).

13. The method according to claim 1, further comprising applying an additional cosmetic agent (M2) to the skin, wherein the additional cosmetic agent (M2) comprises at least one antiperspirant aluminum and/or aluminum zirconium salt in a cosmetically acceptable carrier.

14. A package unit (kit-of-parts), comprising—separately packaged—
a) at least one first container (C1) comprising a cosmetic agent (M1) comprising, in a cosmetically acceptable carrier, at least one phosphate compound and at most about 1.0 wt. % relative to the total weight of the cosmetic agent (M1), antiperspirant aluminum and/or aluminum-zirconium salts, and
b) at least one second container (C2) comprising a cosmetic agent (M2) comprising at least one antiperspirant active ingredient.

15. An antiperspirant cosmetic agent (M1) comprising:
a cosmetically acceptable carrier;
at least one phosphate compound; and
a maximum of about 1.0 weight % of antiperspirant aluminum and/or aluminum zirconium salts.

16. The method of claim 1 wherein:
the at least one phosphate compound has the formula (I)
MₓH_{y}P_{z}O_{3z+1} (I),
where
x and z, each independently of one another, denote integers from about 1 to about 10.

17. The method of claim 16 wherein:
y denotes integers from about 0 to about 20.

18. The method of claim 1 wherein:
the cosmetic agent (M1) comprises the at least one phosphate compound in a total quantity of from about 0.1 to about 40 wt. %, relative to the total weight of the cosmetic agent (M1).

19. The method of claim 1 wherein:
the cosmetic agent (M1) comprises the at least one phosphate compound in a total quantity of from about 1.0 to about 25 wt. %, relative to the total weight of the cosmetic agent (M1).

20. The method of claim 1 wherein:
the cosmetic agent (M1) comprises the at least one phosphate compound in a total quantity of from about 2.0 to about 20 wt. %, relative to the total weight of the cosmetic agent (M1).
